(19) United States
(12) Patent Application Publication
(10) Pub. No.: US 2001/0003145 A1
(43) Pub. Date: Jun. 7, 2001

(54) Judgment method of the brain wave activity and the brain wave activity quantification measurement equipment

(76) Inventors: Akio Mori, Tokyo (JP); Yasuo Saito, Tokyo (JP)

Correspondence Address: FLYNN, THIEL, BOUTELL & TANIS, P.C., 2026 Rambling Road, Kalamazoo, MI, 49008-1699, US

(21) Appl. No.: 09/725,361

(22) Filed: Nov. 29, 2000

(30) Foreign Application Priority Data
Dec. 3, 1999 (JP) 345523/1999

(51) Int. Cl. A61B 5/048

(52) U.S. Cl. 600/544

(57) ABSTRACT

An α wave signal and a β wave signal are separated from a brain wave signal S at the points of the subject's forehead, and under the condition of preset time of a sampling cycle with a settled integration time, a ratio of an integration value of the β wave signal to an integration value of the α wave signal is calculated to obtain the information for judgment of the brain activity. Under the condition of preset time of the sampling cycle with a settled integration time, each integration value of brain wave signal S, said α wave signal and said β wave signal are calculated, then an occurrence ratio of the integration value of the α wave signal to the integration value of the brain signal S is calculated and made to be α% for each sampling cycle, β% is calculated by the same procedure, and the frequency distribution curve of the α% and β% is calculated to obtain the information for judgment of the brain activity. The ratio of β% to α% is calculated for each sampling cycle, then the average values and the frequency distribution curve of the ratio value of β% to α% in a sampling period is calculated to obtain the information for the judgment of the brain activity. According to these results of the information described above, the mind disorder is correctly judged and also the fault of the questionnaire judgment can be compensated.

### TECHNICAL FIELD OF THE INVENTION

The present invention relates to a judgment method of the brain wave activity and the brain wave activity quantification measurement equipment detecting the brain wave signals of humans with either normal the awaking consciousness condition or the resting condition. More detailed, the present invention relates to the judgement method of the brain activity for judging abnormal mental state such as dementia or manic-depressive condition by converting to the numerical value of the brain wave information and the present invention also relates to the brain wave activity quantification measurement equipment for obtaining the information for the brain activity.

1. Prior Art

In operating the system of the nursing care insurance for the elderly, it is very important to judge objectively whether or not the person has a mind disorder such as dementia and to judge objectively the degree of his disease.

In generally, the diagnosis of dementia is done by the operated in the procedure in which the medical specialist has interviews with the dementia persons, asks them the some set questions (e.g. The Hasegawa Scale or The Mental Status Questionnaire in U.S.A.), gets answers and makes a judgement based on the results of the analysis of those answers.

There is also, another procedure that measures the brain waves, separates the α wave (8 to 13 Hz), α wave-(14 to 30 Hz), θ wave (4 to 7 Hz) and δ wave (0.5 to 3.5 Hz) from the said brain waves and judges the degree of a mental disease by the frequency of the brain wave primarily detected.

The procedure of interviewing is problematical in that it is very difficult to judge correctly whether or not the person is in a condition of dementia when he has no answers, he doesn't answer consciously, or he tells a lie.

The procedure of measurement of the brain wave is problematical in that it is difficult to measure correctly because of the patients' fears (especially old persons) concerning the hospital environment and the method of the brain wave which is operated by the electroencephalograph. The medical specialist then must analyze the complicated brain wave.

And, in general, the procedure of measurement of the brain wave is problematical in that it is impossible to judge correctly the degree of the mental disease or to make a pathological diagnosis based on analysis of the electro-encephalogram, so this procedure is used only as an aid in the clinical diagnosis.

The first objection of present invention is to provide a judgment method of brain wave activity which will make it possible to judge and mental disease of manic-depression or the dementia correctly by measuring the brain activity of each person as the objective numerical value in their daily lives.

The second objection of the present invention is to provide the brain wave activity quantification measurement equipment that is small and portable to be able to measure the brain waves of the subjects in the conditions of their daily lives.

The third objection of the present invention is to provide the brain wave activity quantification measurement equipment which will make it possible to measure the brain wave activity correctly without the subjects' feelings of fear, especially for the old dementia patients.

### BRIEF SUMMARY OF THE INVENTION

The present inventors compared the occurrence ratio of α waves and β waves especially in the case of the awaking and resting periods and these waves which were separated from the brain wave. Then we found in the case of the normal persons, the α wave and the β wave are polarized in the awaking and resting periods, but in the case of the patients with mind disorder such as dementia (Hereinafter, it says “the dementia persons”.), the occurrence quantity of the β wave is so little that the α wave and the β wave are not polarized in the periods of awaking and resting and the occurrence ratio of the α wave and the β wave in the period of awaking is similar to that of the normal persons in the period of resting.

The judgment method of the brain wave activity and the brain wave activity quantification measurement equipment according to the present invention was designed based on the discoveries mentioned above.

The procedure described below is necessary to realize the method of the present invention: separating the α waves and the β waves from the brain wave signals that are detected from the subject's forehead points during the sampling time with a settled integration time and calculating the ratio of the integration values of the β wave signals to the integration values of the α signals. Then at each sampling cycle, calculating the integration values of the brain wave signals, the α signals and the β wave signals during the set sampling time with the settled integration time, making the occurrence ratio of the integration value of α signals to the integration value of the brain wave signals to be the α%, making the occurrence ratio of the integration value of β wave signals to the integration value of the brain wave signals to be the β%, then calculating the average value and the frequency distribution curve for α% and β% and the ratio of β% to α% in the measuring sampling period. Then, the results of calculation provide the information for the judgment of the brain activity.

By the methods described above, the problems of the conventional procedure that judges the mental disease by an interview with the persons is solved, and whether or not the person has a mind disorder such as dementia can be judged correctly.

Also, the equipment of the present invention is so small and portable that it is possible to measure the brain activity of the persons in similar condition with their daily lives and it does not need the complicated analysis of the brain wave signals, such as the electro-encephalograph.

Under the conditions described above, tit is possible to measure the brain activity correctly according to the present invention without causing feelings of fear in the persons, especially for older dementia persons.

### BRIEF DESCRIPTION OF DRAWINGS

FIG. 1

A block diagram showing the brain wave activity quantification measurement equipment as a concrete example of the present invention.

FIG. 2

A flowchart showing procedures in the judgment method of the brain wave activity and the brain wave activity quantification measurement equipment.

FIG. 3

Brain wave detection data and calculation data detected from 37 subjects who have the mind disorder of dementia.

FIG. 4

Brain wave detection data and calculation data detected from a normal person in the periods of awaking and resting.

FIG. 5

A point diagram showing the calculation results of each data shown in FIG. 3 and 4; (a) is a point diagram of the dementia persons where the horizontal represents ΣS2 and the vertical represents AW=β/α=ΣΣβ2/ΣΣα2, and (b) is a point diagram of normal person.

FIG. 6

A diagram of the variation per time where the horizontal represents the time (second) and the vertical represents the integration values (ΣS2, Σα and Σβ); (a) is the diagram of variation per time of a particular dementia person (No.19) in the period of awaking, (b) is the diagram of the variation per time of a normal person in the period of awaking, and (c) is a diagram of the variation per time of a normal person in the period of resting.

FIG. 7

A frequency distribution diagram where the horizontal represents the % value and the vertical represents the occurrence frequency P of α% and β%; (a) is the frequency distribution diagram of a particular dementia person (No.19) in the period of awaking, (b) is a frequency distribution diagram of a normal person in the period of awaking, and (c) is the frequency distribution diagram of a normal person in the period of resting.

FIG. 8

A frequency distribution diagram where the horizontal represents β/α and the vertical represents the occurrence frequency P of β/α; (a) is the frequency distribution diagram of a particular dementia person (No.19) in case of the awaking, (b) is the frequency distribution diagram of a normal person in the period of awaking, and (c) is the frequency distribution diagram of a normal person in the period of resting.

FIG. 9

An example showing three different kinds of data of a person with serious dementia (No.8) in the period of awaking under the same condition; (a) is the diagram of the variation per time where the horizontal represents the time (second) and the vertical represents the integration values (ΣS2, Σα and Σβ), (b) is the frequency distribution diagram where the horizontal represents the % value and the vertical represents the occurrence frequency P of α% and β% and (c) is the frequency distribution diagram where the horizontal represents β/α and the vertical represents the occurrence frequency P of β/α.

FIG. 10

An example showing three different kinds of data of a person with moderate dementia (No.18) in the period of awaking under the same condition; (a) is a diagram of the variation per time where the horizontal represents the time (second) and the vertical represents the integration values (ΣS2, Σα and Σβ), (b) is a frequency distribution diagram where the horizontal represents the % value and the vertical represents the occurrence frequency P of α% and β%, and (c) is a frequency distribution diagram where the horizontal represents β/α and the vertical represents the occurrence frequency P of β/α.

FIG. 11

An example showing three different kinds of data of a person with mild dementia (No.12) in the period of awaking under the same condition; (a) is a diagram of the variation per time where the horizontal represents the time (second) and the vertical represents the integration values (ΣS2, Σα and Σβ), (b) is a frequency distribution diagram where the horizontal represents the % value and the vertical represents the occurrence frequency P of α% and β%, and (c) is a frequency distribution diagram where the horizontal represents β/α and the vertical represents the occurrence frequency P of β/α.

FIG. 12

A frequency distribution diagram where the horizontal represents β/α and the vertical represents the occurrence frequency P of β/α, showing an example diagram where the data of the serious dementia person (No.8), the moderate dementia person (No.8) and the mild dementia person (No.12) are overlapped and compared

FIG. 13

A distribution map where the horizontal represents β/α and the vertical represents the number P of the dementia persons within 37 dementia persons at a care facility.

### DETAILED DESPRIPTION

The brain waves of human beings are categorized as α (8-13 Hz), β wave (14-30 Hz), θ wave (4-7 Hz), δ wave (0.5 -3.5 Hz) and so on by the frequency.

The α occurs dominantly when the subjects are in a resting condition (but, it is not sleep) or in a just waking-up condition (Hereinafter, it says, “the resting”). The β wave occurs dominantly when the subjects are in a thinking activity condition when he is awaking and in the clearly waking-up condition (Hereinafter, it says “the awaking”).

The θ wave occurs dominantly when the subjects are in a drowsy condition at the beginning of sleep.

The δ wave occurs dominantly when the subjects are in a deep sleep condition.

The present inventor compared the occurrence ratio of the α wave and the β wave especially in case of awaking and resting periods. And the inventor found that in the normal person, the brain waves in case of awaking and resting periods are polarized, but in the person who has a mental disease such as dementia (Hereinafter, it says “dementia persons”), the occurrence quantity of the β wave is so little that the α and the β wave are not polarized in case of the awaking and the resting, and their occurrence ratio of the α wave and the β wave in case of the awaking is similar with that of the normal persons in case of the resting.

The primary principle of the present invention is explained as follows:

The α signal is defined as the criteria of digitalization in the present invention, because the occurrence quantity of the α wave signal is treated as the criteria signal for observing the mental condition.

When the compound brain wave signal which is detected from the subject and input to the equipment according to the present invention is made to be S (hereinafter, it says “brain signal S”), S is expressed as the following formula;

S=θwave+αwave+βwave

In the present invention, S is the brain wave signal composed of the brain wave signals of three kinds (θ wave, α and β wave), at least , so that each signal of the θ wave, the α and the β wave is digitized by the procedure described as follows.

(1) An exclusive electrode is attached on the head of the subject to conduct the brain wave signal from the subject.

As the original signal of this brain wave is a very small signal which is about 10 μV-100 μV, the signal is amplified to about 1 V by the height-gain amplifier and the signal S is filtered out of the 3-30 Hz through a filter-amplifier. Then, the signal S is separated in each signal of the θ wave, the α and the β wave by the filters, and each signal which is separated from the signal S is made to be θ1, α1 and β1 respectively.

(2) The signal S and each signal of the θ wave, the α and the β wave are converted to the digitized signal by the analogue-to-digital converter respectively. And each converted signal is made to be S2, θ2, α2 and β2.

(3) The digitized signals of S2, θ2, α2 and β2 are integrated respectively at a suitable set integration time. In the present invention, the integration time and the sampling time are set at 3 seconds and that time is made to be the sampling integration time. The integrated signals are made to be Σ S2, Σθ2, Σα2 and Σβ2 respectively.

(4) The occurrence ratio (%) of the each integration values of θ2, α2 and β2 to the integration value of signal S2 are respectively calculated. Said occurrence ratio is θ%=Σθ2/Σ S2, α%=Σα2/Σ S2, β%=Σβ2/Σ S2. Besides, by calculating the occurrence ratio of the brain wave signal, the problem that the brain waves have the individual differences by the deviation of amplitude are resolved.

(5) As the mental activity of human being is continuous, the average value of each signal is respectively calculated in the sampling period T (the sampling integration time t×the number of sampling cycle N) to preserve the accuracy of the analysis. For example, when the sampling integration time t is 3 seconds and the number of sampling cycles N are 100 times, the average value is calculated based on the condition that the sampling period T is longer than 5 minutes. When the average values are made to be θ3, α3 and β3 respectively, θ3=Σθ%/N, α3=Σα%/N and β3=Σβ%/N are calculated.

(6) Using the average values θ3, α3 and β3 that are provided with the operations described above, the awaking index AW=β3/α3 and the drowsing index SL=θ3/α3 are calculated.

(7) The awaking index AW and the drowsing index SL can be obtained by the following formula:

AW=β/α3=(Σβ%/N)/(Σα%/N)=Σβ%/Σα%=Σ(Σβ2/ΣS2)/Σ(Σα2/ΣS2)=ΣΣβ2/ΣΣα2

AL=θ3/α3=(Σθ%/N)/(Σα%/N)=Σθ%/ Σα%=Σ (Σθ2/ΣS2)/Σ(Σα2/ΣS2)=ΣΣθ2/ΣΣα2

(8) Also, by displaying the frequency distribution diagrams of θ%, α% and β% in the sampling period T (the sampling integration time t×the number of sampling cycles N), the relationship of each frequency band can be displayed on the diagrams. According to these frequency distribution diagrams, it is recognized that frequency band of the normal persons and the dementia persons is very conspicuously different, the dementia state and the mind disorder such as the manic-depression can be distinguished by said AW, and the information for the brain activity in the drowsing is obtained from said SL.

The function of the present invention is described as follows.

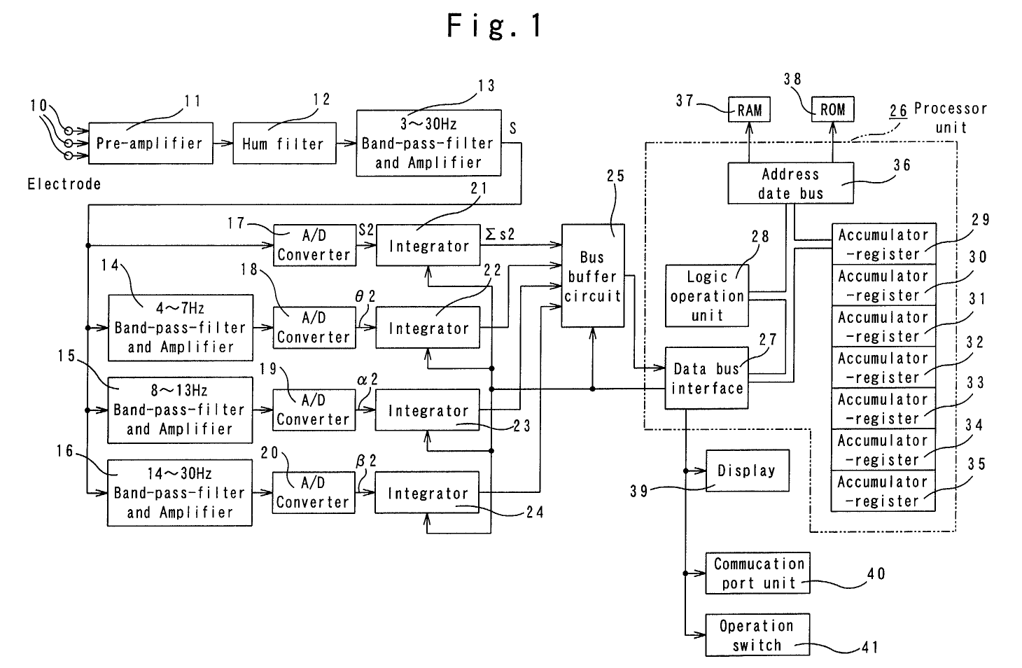

In FIG. 1, 10 represents the plurality of the brain wave electrode attached on the subject's forehead. The brain wave electrode 10 is connected to the band-pass-filter and amplifier 13 extracts the condition of the brain wave signal of the θ wave (4 -7 Hz), the α (8-13 Hz) and the β wave (14-30 Hz) through the pre-amplifier 11 and the hum filter 12. The band-pass-filter and amplifier 13 is connected to the band-pass-filter and amplifier 14, 15 and 16.

Then, the band-pass-filter and amplifier 13 is connected to the A/D converter 17 and the integrator 21, the band-pass-filter and amplifier 14 is connected to the A/D converter 18 and the integrator 22, the band-pass-filter and amplifier 15 is connected to the A/D converter 19 and the integrator 23, and the band-pass-filter and amplifier 16.is connected to the A/D converter 20 and the integrator 24, to connect the bus buffer circuit 25.

The bus buffer circuit 25 is connected to the data bus interface 27 of the processor unit 26 which consists of a microcomputer. The processor unit 26 comprises the logic operation unit 28, accumulator-registers 29, 30, 31, 32, 33, 34, 35 and the address data bus 36. The RAM 37 and the ROM 38 are connected with said address data bus 36 and the display 39, the communication port unit 40 and the operation switch 41 are connected with said data bus interface 27.

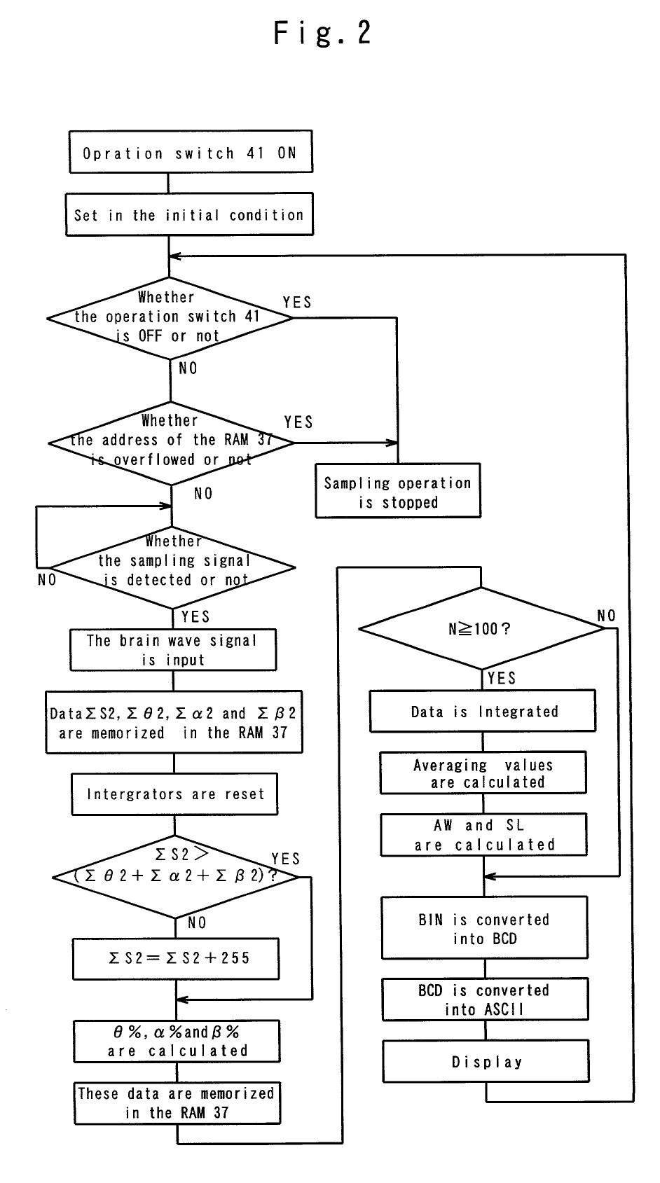

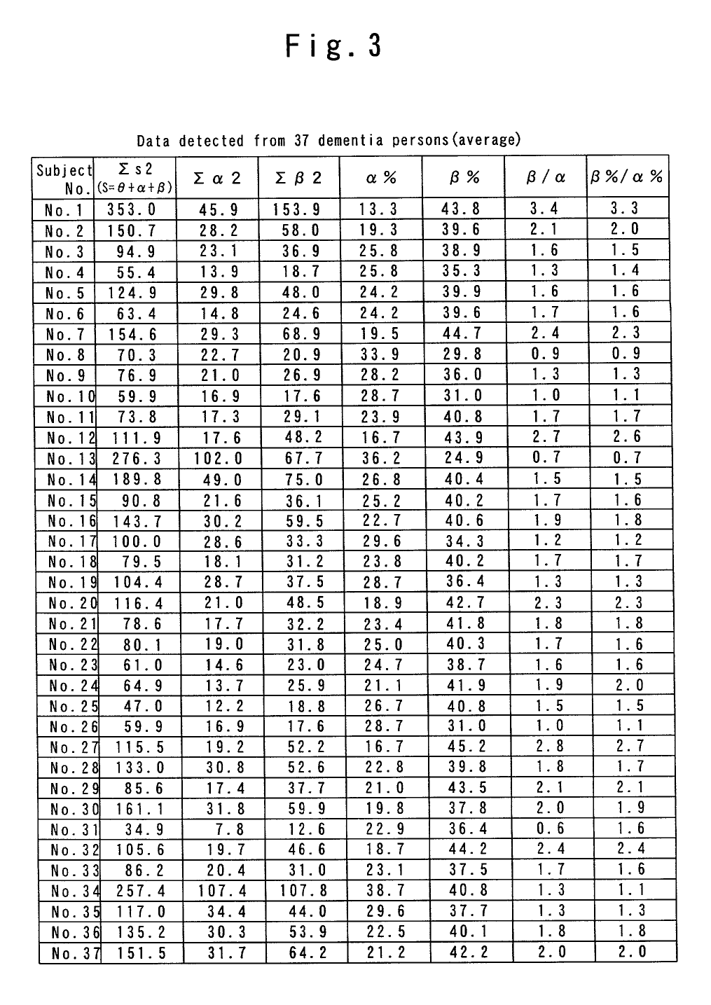

The operations of the present invention are explained as follows according to FIG. 1 and FIG. 2.

(1) In FIG. 2, the equipment according to the present invention shown in FIG. 1 begins the operation by making the operation switch 41 ON, and all the circuit units are set in the initial condition. When the answer of the question whether the address ADN of the RAM 37 is overflowed or not is NO, and when the answer of the question whether the sampling signal is detected or not is YES, the brain wave signal is inputted.

The said brain wave signal is conducted by attaching exclusive electrode 10 to the head of the subject. As the original signal of this brain wave is a small signal which is about 10 μV-100 μV, the signal is amplified to about 1 V in the pre-amplifier 11, the noise of the brain wave is avoided in the hum filter 12 of 50/60 Hz, and the signal S of 3-30 Hz is abstracted in the band-pass-filter and amplifier 13 to output. Then, each signal θ1, α1 and β1 of the θ wave, α wave and the β wave is output from the signal S by the band-pass-filter and amplifier 14, 15 and 16.

(2) The signal S and each signal θ1, α1 and β1 are converted to the digitized signal by the A/D converter 17, 18, 19 and 20 respectively. The digitized signals are made to be S2, θ2, α2 and β2 respectively.

(3) The digitized signal S and each digitized signal θ2, α2 and β2 are integrated in the integrators 21, 22, 23 and 24 at the set time of about 1 to 10 seconds (3 seconds of sampling cycle, in the present sample), and these signals are converted into the digitized integration values (binary 8 bits) ΣS2, Ση2, Σα2 and Σβ2.

These integration signals of binary 8 bits are transferred to the processor unit 26 through the bus buffer circuit 25, call the RAM address ADN by control of the logic operation unit 28, memorized sequentially to the RAM 37 from the address ADN through the accumulator-register 29 to 35, and call the number of sampling cycles N to add 1 to the said number of times N.

The integrators 21, 22, 23 and 24 are reset to the initial condition. The said integration time is controlled by the processor unit 26.

(4) The operation to decide whether ΣS2>(Σθ2+Σα2+Σβ2) ? and the operation of ΣS2=ΣS2+255 are necessary because the memory is 8 bits (=256), so these operations are unnecessary if the memory is larger than 8 bits.

The occurrence ratio (%) of each signal θ, α and β to the signal S is calculated. The occurrence ratio of the integration values is θ%=Σθ2/ΣS2, α%=Σα2/ΣS2, β%=Σβ2/ΣS2 respectively. These data are memorized in the RAM 37.

(5) As the mental activity of humans is continuous, the average value is calculated in the sampling period T (a unit of the sampling integration time t×the number of sampling cycles N) to reserve the accuracy of the analysis. For example, when the sampling integration time t is 3 seconds and the number of sampling cycles N is 100 times, the average value is calculated based on the condition that the sampling period T is longer than 5 minutes. When the answer to the question whether the sampling times N ≧100 or not is NO, the average is only displayed without being calculated.

(6) When the answer to the question whether N≧100 or not is YES, Σθ%, Σα% and Σβ% are calculated in the data integrating operation, and each average θ3=Σθ%/N, α3=σα%/N and β3=Σβ%/N is calculated.

(7) Then the awaking index AW=β3/α3 and the drowsing index SL=θ3/α3 are obtained from θ3, α3 and β3 by calculation.

(8) The awaking index AW and the drowsing index SL can be obtained by the following formula:

AW=β3/α3=(Σβ%/N)/(Σα%/N)=Σβ%/Σα%=Σ(Σβ2/ΣS2)/Σ(Σα2/ΣS2)=ΣΣβ2/ΣΣα2

AL=θ3/α3=(Σθ%/N)/(Σα%/N)=Σθ%/Σα%=Σ(Σθ2/ΣS2)/Σ(Σα2/ΣS2)=ΣΣθ2/ΣΣα2

(9) For displaying the data, the binary data is converted into the data or the ASCII code and memorized to the temporary storage area of the RAM 37.

(10) By the operations explained above, the occurrence scatter diagram and other characteristic diagrams are obtained, each diagram or each result of the calculation θ%, α%, β%, AW, SL and ΣS2, Ση2, Σα2, Σβ2 and so on are displayed in the display 39, and these results are output to another view of equipment or the like from the communication port unit 40.

Then, the examples of the concrete data of the normal persons and the dementia persons which are analyzed in the equipment according to the present invention is explained as follows;

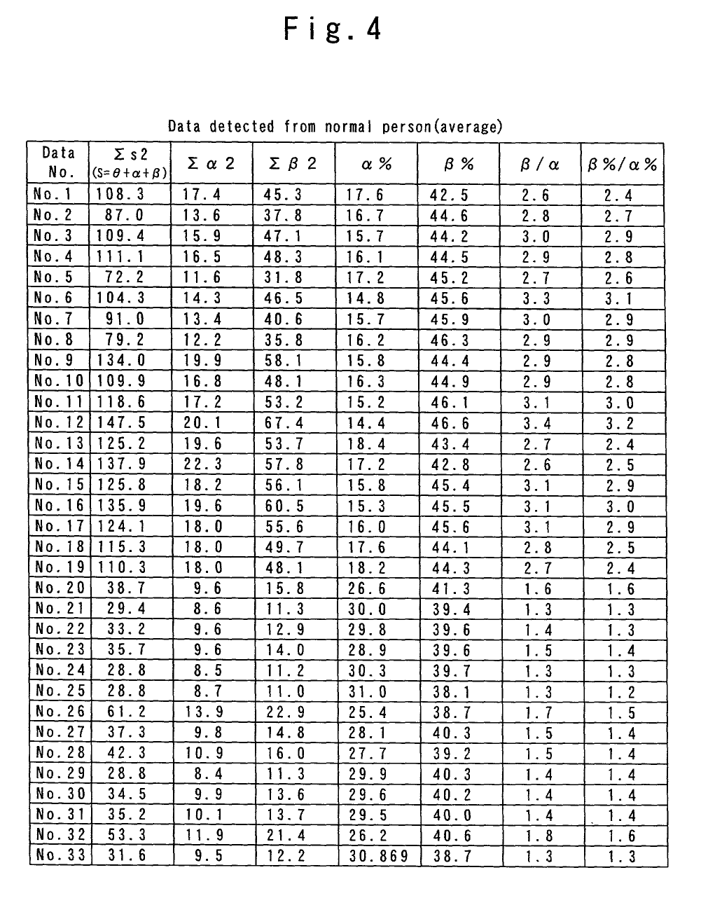

Concerning the normal person (age 69, male), the data are gathered using the brain wave activity quantification measurement equipment according to the present invention in the condition of the sampling integration time t (3 seconds)×the sampling cycles N (120 times)=the sampling period T (6 minutes), and the frequency distribution diagram of each α% and β% at each sampling cycle which are at work time in the periods of awaking and resting making adjustment in the calculations when the eyes are opened and shut. FIG. 4 shows the data of the normal persons; the data of No.1- No.19are the data at work time in the period of awaking, the data of No.20- No.33 are the data at the rest time with opening eyes in the period of awaking, and each column of ΣS2, Σα2, Σβ2, α%, β%, β/α and β%/α% about each data number is a calculation result.

The analysis examples of the normal persons are explained as follows.

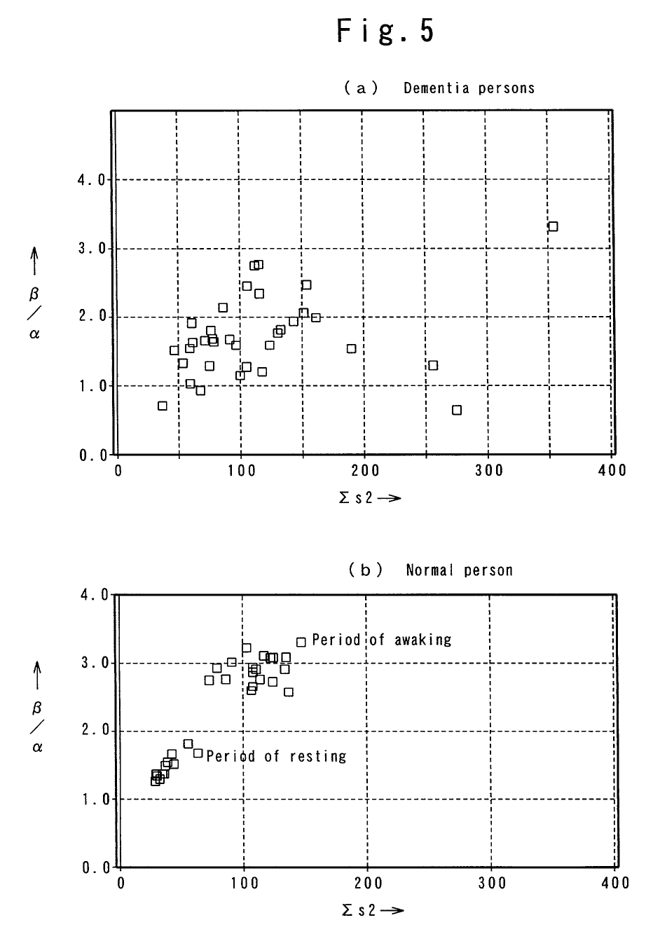

FIG. 5(b) shows the point diagram of the normal persons where the horizontal represents Σ S2 and the vertical represents AW=β/α=ΣΣ↑2/ΣΣα2

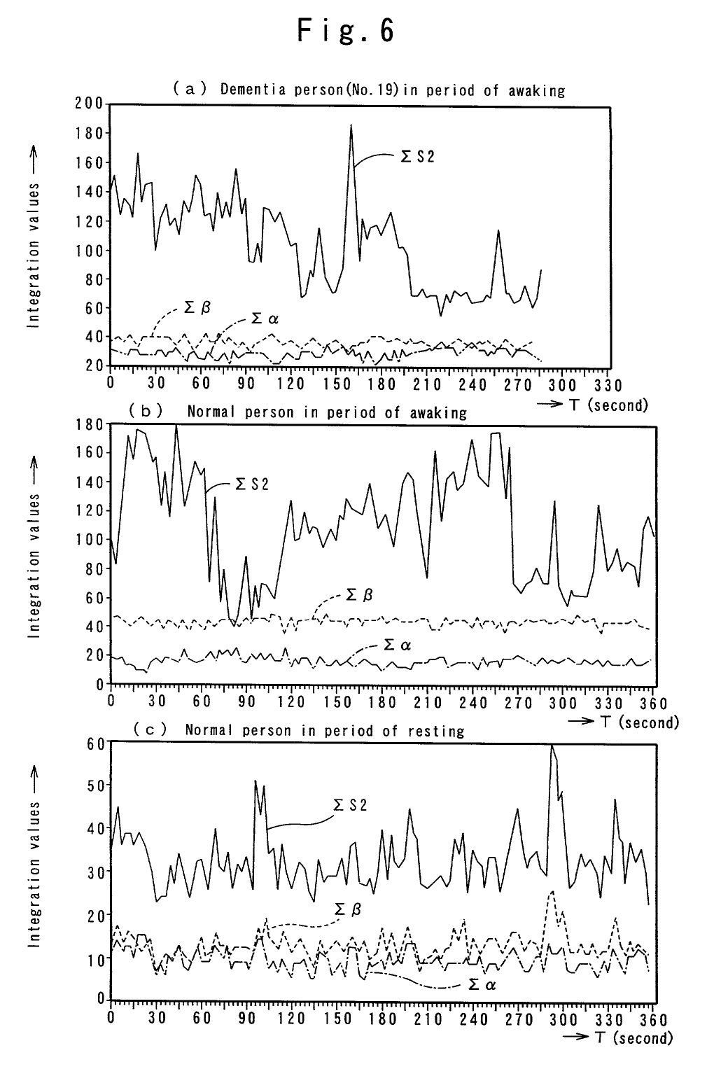

FIG. 6(b) shows the diagram of the variation per time of a normal person in the period of awaking where the horizontal represents the time (second) and the vertical represents the integration values (ΣS2, Σα and Σβ), and (c) shows the diagram of the variation per time of a normal person while resting.

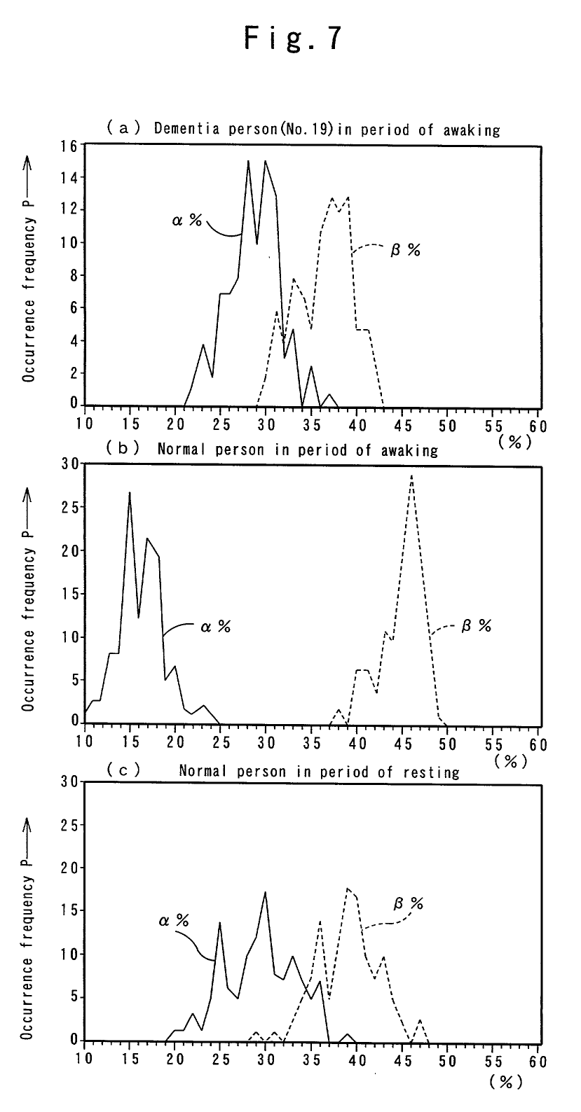

FIG. 7(b) shows the frequency distribution diagram of a normal person in the period of awaking where the horizontal represents the % value and the vertical represents the occurrence frequency P of α% and β%, and (c) shows the frequency distribution diagram of normal person while resting.

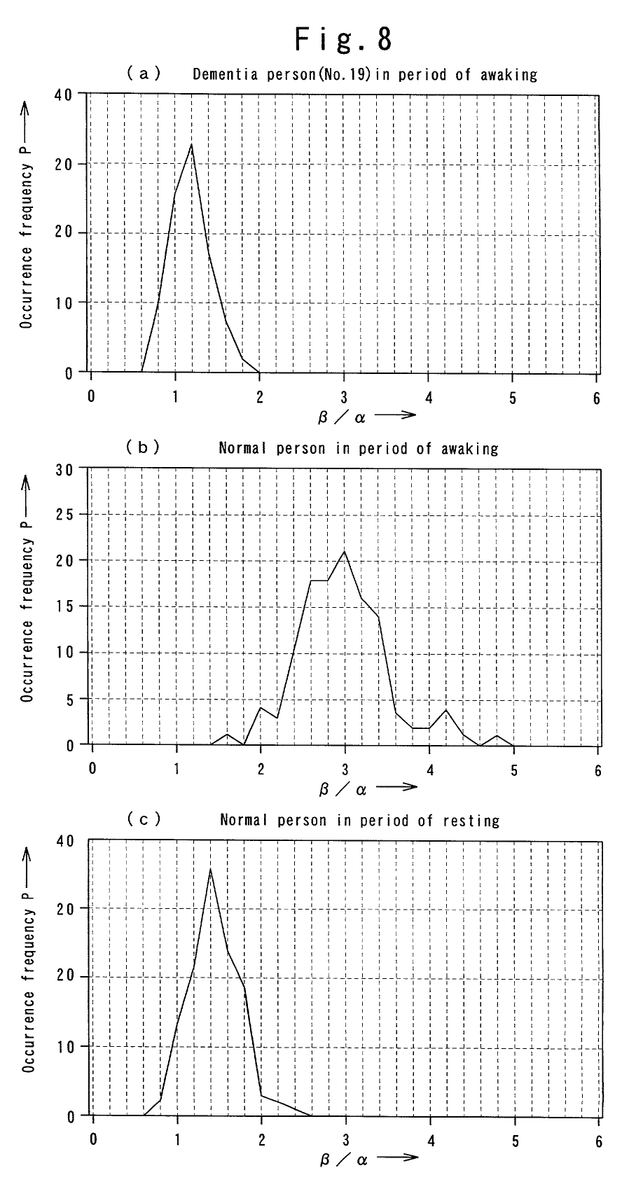

FIG. 8(b) shows the frequency distribution diagram of a normal person in the period of awaking where the horizontal representsβ/α and the vertical represents the occurrence frequency P of β/α, and (c) shows the frequency distribution diagram of a normal person while resting.

In these characteristic diagrams, according to FIG. 7(b), the average occurrence ratio of β% in the period of awaking of the normal person is about 45%, and that of α% is about 16%. That is, the occurrence ratio of β% is almost three times more than that of α%. And this result is obvious by the characteristic diagram of

FIG. 8(b). Also, according to FIG. 7(c), the average occurrence ratio of β% in the period of awaking of the normal person is about 40%, and that of α% is about 28%. That is, the occurrence ratio of β% is almost 1.4. times more than that of α%. And this result is obvious by the characteristic diagram of FIG. 8 (c).

According to FIG. 5(b), in the point diagram of AW value to the average of the integration value of the signal S (=ΣΣS2/N), it is found that when the normal person is in the period of resting, the average (=ΣΣS2/N) becomes less than 100 and the AW becomes less than 2.0 and when the normal person is in the period of awaking, the average (=ΣΣS2/N) becomes more than 70 and the AW becomes more than 2.0, and it is also found that the distribution of averages in the periods of awaking and resting are separated obviously. That is, the average index AW which is the ratio of the β to the a of the normal person in case of the awaking is more than 2.5 and the average index AW that is the ratio of the β to the α of the normal person in the period of resting is within 1.3-1.8. Also, the condition of the brain activity of the normal person in his daily life is separated by the boundary line of the AW value 2.0 which is to the average brain wave integration (=ΣΣS2/N).

Next, about the 37 of the older dementia persons, the data in the interview to judge (the question contents are identical about all the members) Next, data about the 37 older dementia persons which are obtained in an interview examination in which everyone is asked the same questions concerning their mental status is collected in the brain wave activity quantification measurement equipment according to the present invention and then this. data is classified. For the dementia persons, the mental effort required during the interview is equal to the thinking work for the normal persons.

FIG. 5(a) shows a point diagram in the period of awaking of each of 37 dementia persons in a case similar to FIG. 5(b).

FIG. 6(a) shows the diagram of the variation per time in the period of awaking of a particular dementia person (No.19) in a case similar to FIG. 5(b).

FIG. 6(a) shows a frequency distribution diagram in the period of awaking of a particular dementia person (No.19) in a case similar with FIG. 5(b).

In these figures, according to FIG. 7(a), the average occurrence ratio of β% in the period of awaking of the dementia person is about 36%, and that of α% is about 28%. That is, the occurrence ratio of β% is almost 1.3 times more than that of α% and that ratio is almost same with that of the normal person in the period of resting. And this result is similar to the condition of the normal person in the period of just awaking and it is obvious by the characteristic diagram of FIG. 8(a).

According to FIG. 5(a), it is found that the condition of whole brain activity is lively(active) when the average brain wave integration (=ΣΣS2/N) is more than 100 and the AW is less than 1.0, but that is in the condition which is completely unrelated to conscious activity and which is indifferent to stimulation from outside.

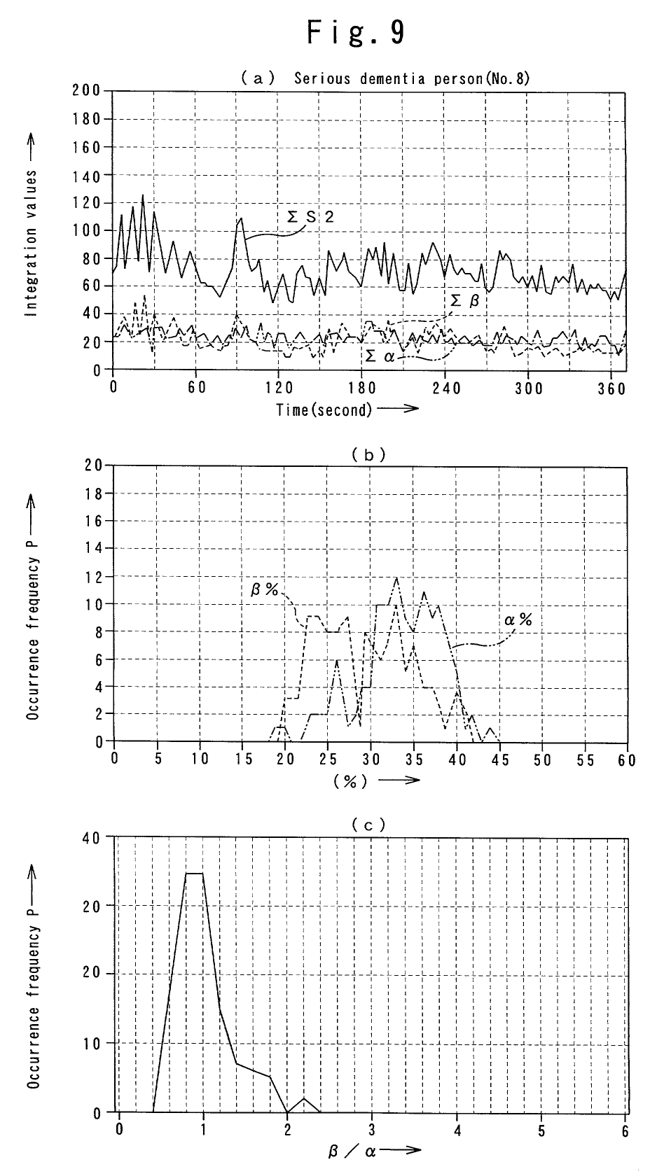

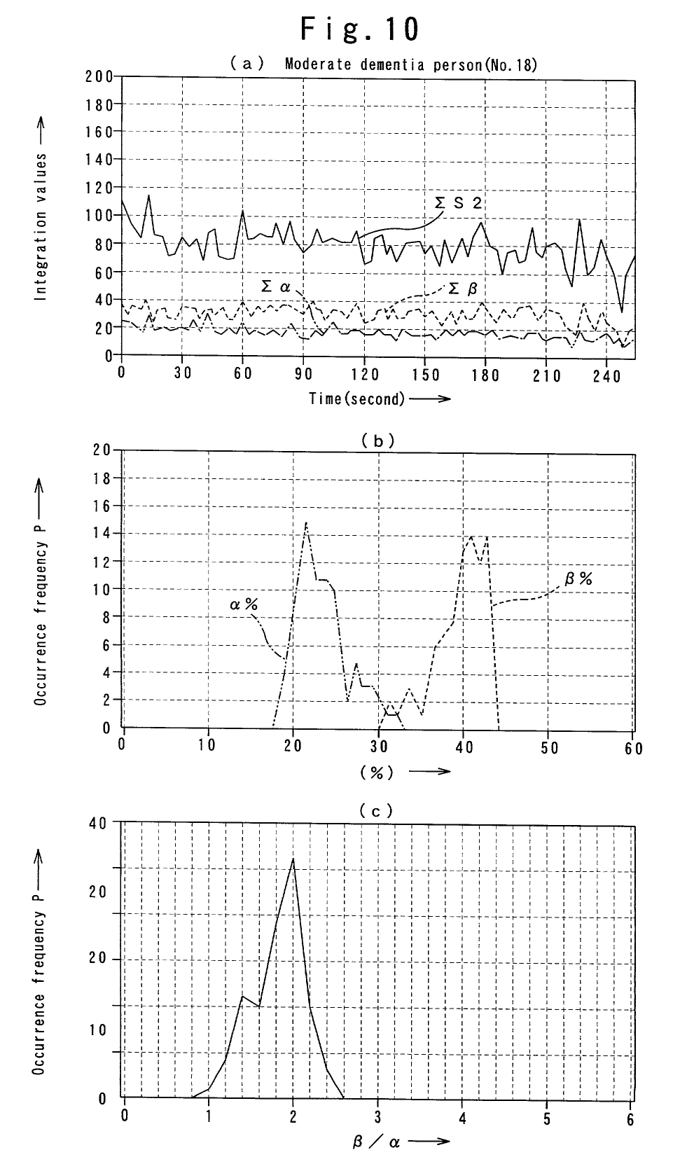

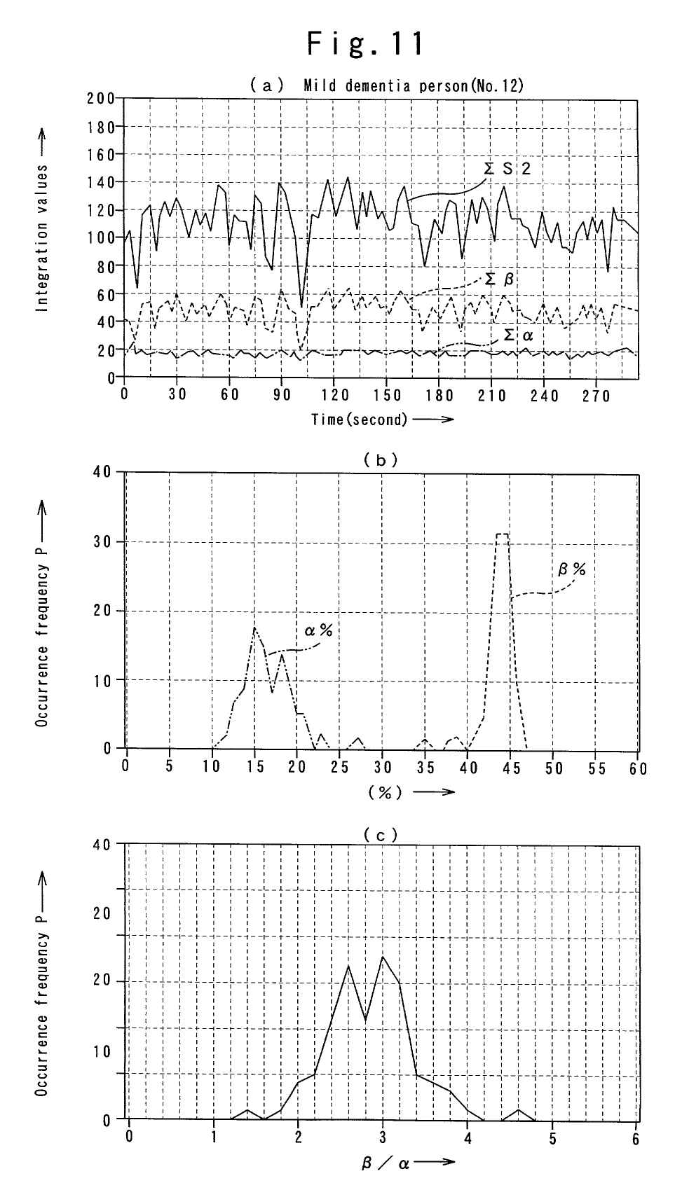

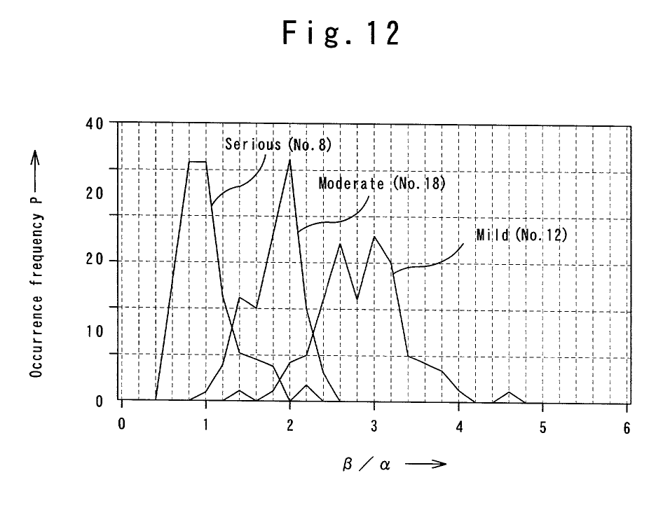

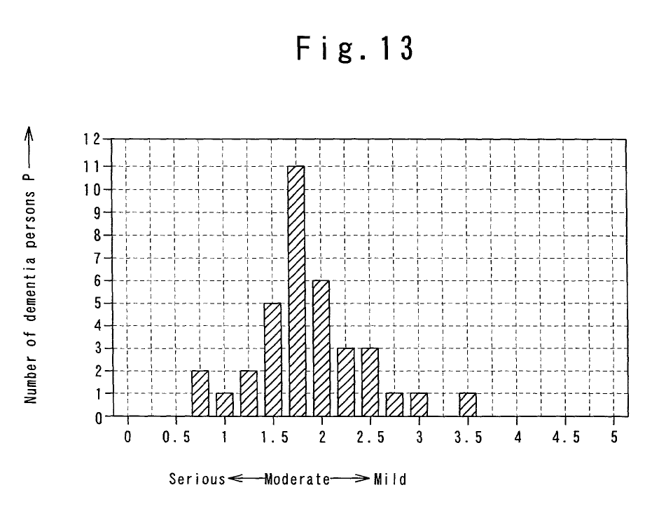

FIG. 9 shows the three different kinds data of the serious dementia person (No.8) in the period of awaking under the same condition. In FIG. 9, (a) is a diagram of the variation per time where the horizontal represents the time (second) and the vertical represents the integration values (ΣS2, Σα and Σβ), (b) is a frequency distribution diagram where the horizontal represents the % value and the vertical represents the occurrence frequency P of α% and β%, and (c) is a frequency distribution diagram where the horizontal represents β/α and the vertical represents the occurrence frequency P of β/α.

FIG. 10 shows the example showing three different kinds data of the moderate dementia person (No.18) in the period of awaking under the same condition. In FIG. 10, (a) is a diagram of the variation per time where the horizontal represents the time (second) and the vertical represents the integration values (ΣS2, Σα and Σβ), (b) is a frequency distribution diagram where the horizontal represents the % value and the vertical represents the occurrence frequency P of α% and β%, and (c) is a frequency distribution diagram where the horizontal represents β/α and the vertical represents the occurrence frequency P of β/α.

FIG. 11 shows example showing three different kinds data of the person with mild dementia (No.12) in the period of awaking under the same condition. In FIG. 11, (a) is a diagram of the variation per time where the horizontal represents the time (second) and the vertical represents the integration values (ΣS2, Σα and Σβ), (b) is a frequency distribution diagram where the horizontal represents the % value and the vertical represents the occurrence frequency P of α% and β%, and (c) is a frequency distribution diagram where the horizontal represents β/α and the vertical represents the occurrence frequency P of β/α.

FIG. 12 shows a frequency distribution diagram where the horizontal represents β/α and the vertical represents the occurrence frequency P of β/α, showing the example diagram that the data of the person with serious dementia (No.8), the person with moderate dementia (No.8) and the person with mild dementia (No.12) are overlapped and compared. FIG. 12 shows obviously the degree of dementia in the comparison.

FIG. 13 shows a distribution map where the horizontal represents β/α and the vertical represents the number p of the dementia persons within 37 dementia persons at the particular facility:

What is claimed is:

1. A judgment method of the brain wave activity characterized in that the brain wave signals during the sampling time in the subjects are detected, α wave signals and β wave signals are separated from the brain wave signals, the ratio with the β wave signals to the α signals are calculated, and brain wave activities are judged based on these calculation results.

2. A brain wave activity quantification measurement equipment characterized in that comprising the separators separating α signals and β wave signals from the brain wave signals during sampling time in the subj ects, and the calculator calculating the ratio with the β wave signals to the α signals to obtain the information for judgement of the brain wave activity.

3. A judgment method of the brain wave activity characterized in the brain wave signals θ wave signals, α-wave signals and β wave signals during sampling time in the subjects are detected, the α signals and the β wave signals are separated from the brain wave signals, the integration values of the brain wave signals, the integration values of the α signals and the integration values of the β wave signals in the sampling time are integrated, the occurrence ratio of the α signals to the integration values of the brain wave signals is made to be α%, the occurrence ratio of the β wave signal to the integration values of the brain wave signals is made to be β%, the ratio of β% to α% is calculated, and the brain wave activity is judged based on these calculation results.

4. A brain wave activity quantification measurement equipment characterized in that comprising a detection device detecting brain wave signals containing θ wave signals, α wave signals and β wave signals during sampling time in the subjects, the separators separating the ce wave signals and the β wave signals from the brain wave signals, the integrators integrating the integration values of the brain wave signals, the integration values of the α signals and the integration values of the β wave signals, and the calculator making the occurrence ratio of the α wave signals to the integration values of the brain wave signals to be α%, making the occurrence ratio of the β wave signals to the integration values of the brain wave signals to be β% and calculating the ratio of β% to α% to obtain the information for judgement of the brain wave activity.

5. A judgment method of the brain wave activity characterized in that brain wave signals containing θ wave signals, α wave signals and β wave signals during sampling time in the subjects are detected, the α wave signals and the β wave signals are separated from the brain wave signals, the integration values of the brain wave signals, the integration values of the α wave signals and the integration values of the β wave signals in the sampling time are integrated, the occurrence ratio of the α signals to the integration values of the brain wave signals is made to be α%, the occurrence ratio of the α wave signal to the integration values of the brain wave signals is made to be β%, the variation in characteristics of β% and α% per sampling time during the sampling period is calculated, and the brain wave activity is judged based on these changes in characteristics.

6. A brain wave activity quantification measurement equipment characterized in that comprising a detection device detecting brain wave signals containing 0 wave signals, α wave signals and β wave signals during sampling time in the subjects, the separators separating the α signals and the β wave signals from the brain wave signals, the integrators integrating the integration values of the brain wave signals, the integration values of the α signals and the integration values of the β wave signals, and a calculator making the occurrence ratio of the α wave signals to the integration values of the brain wave signals to be α%, making the occurrence ratio of the β wave signal to the integration values of the brain wave signals to be β% and calculating the variations in characteristics of β% and α% per sampling time during the sampling period is calculated to obtain the information for judgement of the brain wave activity.

7. A judgment method of the brain wave activity characterized in that brain wave signals containing θ wave signals, α wave signals and β wave signals during sampling time in the subjects are detected, the α signals and the β wave signals are separated from the brain wave signals, the integration values of the brain wave signals, the integration values of the α signals and the integration values of the β wave signals are integrated, the occurrence ratio of the α signals to the integration values of the brain wave signals is made to be α%, the occurrence ratio of the β wave signals to the integration values of the brain wave signals is made to be β%, the distribution of the occurrence frequency of α% and β% is calculated, and the brain wave activity is judged based on this distribution of the occurrence frequency.

8. A brain wave activity quantification measurement equipment characterized in that comprising a detection device detecting brain wave signals containing θ wave signals, ar wave signals and β wave signals during sampling time in the subjects are detected, the separators separating the α signals and the β wave signals from the brain wave signals, the integrators integrating the integration values of the brain wave signals, the integration values of the α wave signals and the integration values of the β wave signals, and a calculator making the occurrence ratio of the α wave signals to the integration values of the brain wave signals to be α%, making the occurrence rate of the β wave signal to the integration values of the brain wave signals to be β% and calculating the distribution of the occurrence frequency of α% and β% to obtain the information for judgement of the brain wave activity.

9. A judgment method of the brain wave activity characterized in that brain wave signals containing θ wave signals, α signals and β wave signals during sampling time in the subjects are detected, the α signals and the β wave signals are separated from the brain wave signals, the integration values of the α wave signals and the integration values of the β wave signals are integrated, the integration ratio of the integrated β wave signals to the integrated α wave signals is calculated, the distribution of the occurrence frequency of the integration ratio is calculated, and the brain wave activity is judged based on this distribution of the occurrence frequency of the integration ratio.

10. A brain wave activity quantification measurement equipment characterized in that comprising a detection device detecting brain wave signals containing θ wave signals, α wave signals and β wave signals during sampling time in the subjects, the separators separating the α wave signals and the β wave signals from the brain wave signals, the integrators integrating the α wave signals and the β wave signals, a calculator calculating the integration ratio of the integrated β wave signals to the integrated α wave signals, a contour device counting the distribution of the occurrence frequency of the integration rate.

11. A brain wave activity quantification measurement equipment characterized in that comprising an amplifier extracting brain wave signals containing the dominant brain wave signals and the separators separating the α wave signals and the β wave signals from the brain wave signals, an A/D converter digitizing the brain wave signals, the α wave signals and the β wave signals that are extracted, the integrator integrating in the sampling integration time the brain wave signals, the α wave signals and the β wave signals which are converted by the A/D converter, the calculator calculating the ratio of the integration values of the β wave signals to the integration values of the α wave signals, calculating the occurrence ratio of the integration value of the α wave signals to the integration value of the wave signals (α%), calculating the occurrence ratio of the integration value of the β wave signals to the integration value of the wave signals (β%) and calculating the ratio with β% to α% to obtain the information for judgement of the brain wave activity, the m.emory memorizing the calculation program and the results of the calculating, and the display displaying the results of calculation.

12. A brain wave activity quantification measurement equipment as claimed in claim 5, characterized in that comprising a programming device calculating the integration values ΣS2, ΣS α2, ΣSβ, Σα2/ΣS2=α%, Σβ2/ΣS2=β% that are integrated in set sampling integration time t, calculating the average values of Σα%/N=α3, Σβ%/N=β3, β3/α3=AW(awaking index) during the sampling period T included the sampling cycles N, and these calculations operated after the digitized procedures of the brain wave signals S, the α wave signals and the β wave signals to obtain the information for judgement of the brain wave activity.

13. A judgment method of the brain wave activity as claimed in claim 1, 3, 5, 7 and 9, characterized in that the information for judgement of the brain wave activity is applied to the diagnosis help information of dementia and other mental disorders.

14. A judgment method of the bain wave activity characterized in that the brain wave signals during the sampling time in the subjects are detected, α wave signals and β wave signals are separated from the brain wave signals, the ratio with the β wave signals to the a wave signals are calculated, and brain wave activities are judged based on these calculation results.

15. A brain wave activity quantification measurement equipment characterized in that comprising the separators separating α wave signals and β wave signals from the brain wave signals during sampling time in the subjects, and the calculator calculating the ratio with the β wave signals to the α wave signals to obtain the information for judgment of the brain wave activity.

16. A judgment method of the brain wave activity characterized in the brain wave signals θ wave signals, α-wave signals and β wave signals during sampling time in the subjects are detected, the α wave signals and the β wave signals are separated from the brain wave signals, the integration values of the brain wave signals, the integration values of the α wave signals and the integration values of the β wave signals in the sampling time are integrated, the occurrence ratio of the α wave signals to the integration values of the brain wave signals is made to be α%, the occurrence ratio of the β wave signal to the integration values of the brain wave signals is made to be β%, the ratio of β% to α% is calculated, and the brain wave activity is judged based on these calculation results.

17. A brain wave activity quantification measurement equipment characterized in that comprising a detection device detecting brain wave signals containing θ wave signals, α wave signals and β wave signals during sampling time in the subjects, the separators separating the α wave signals and the P wave signals from the brain wave signals, the integrators integrating the integration values of the brain wave signals, the integration values of the α wave signals and the integration values of the β wave signals, and the calculator making the occurrence ratio of the α wave signals to the integration values of the brain wave signals to be α%, making the occurrence ratio of the β wave signals to the integration values of the brain wave signals to be β% and calculating the ratio of β% to α% to obtain the information for judgment of the brain wave activity.

18. A judgment method of the brain wave activity characterized in that brain wave signals containing 0 wave signals, α wave signals and β wave signals during sampling time in the subjects are detected, the α wave signals and the β wave signals are separated from the brain wave signals, the integration values of the brain wave signals, the integration values of the α wave signals and the integration values of the β wave signals in the sampling time are integrated, the occurrence ratio of the α wave signals to the integration values of the brain wave signals is made to be α%, the occurrence ratio of the β wave signal to the integration values of the brain wave signals is made to be β%, the variation in characteristics of β% and α% per sampling time during the sampling period is calculated, and the brain wave activity is judged based on these changes in characteristics.

19. A brain wave activity quantification measurement equipment characterized in that comprising a detection device detecting brain wave signals containing θ wave signals, α wave signals and P wave signals during sampling time in the subjects, the separators separating the α wave signals and the β wave signals from the brain wave signals, the integrators integrating the integration values of the brain wave signals, the integration values of the α wave signals and the integration values of the β wave signals, and a calculator making the occurrence ratio of the α wave signals to the integration values of the brain wave signals to be α%, making the occurrence ratio of the β wave signal to the integration values of the brain wave signals to be β% and calculating the variations in characteristics of β% and α% per sampling time during the sampling period is calculated to obtain the information for judgment of the brain wave activity.

20. A judgment method of the brain wave activity characterized in that brain wave signals containing 0 wave signals, α wave signals and β wave signals during sampling time in the subjects are detected, the α wave signals and the β wave signals are separated from the brain wave signals, the integration values of the brain wave signals, the integration values of the α wave signals and the integration values of the β wave signals are integrated, the occurrence ratio of the α wave signals to the integration values of the brain wave signals is made to be α%, the occurrence ratio of the β wave signals to the integration values of the brain wave signals is made to be β%, the distribution of the occurrence frequency of α% and β% is calculated, and the brain wave activity is judged based on this distribution of the occurrence frequency.

21. A brain wave activity quantification measurement equipment characterized in that comprising a detection device detecting brain wave signals containing θ wave signals, α wave signals and P wave signals during sampling time in the subjects are detected, the separators separating the α wave signals and the β wave signals from the brain wave signals, the integrators integrating the integration values of the brain wave signals, the integration values of the α wave signals and the integration values of the β wave signals, and a calculator making the occurrence ratio of the α wave signals to the integration values of the brain wave signals to be α%, making the occurrence rate of the β wave signal to the integration values of the brain wave signals to be β% and calculating the distribution of the occurrence frequency of α% and β% to obtain the information for judgment of the brain wave activity.

22. A judgment method of the brain wave activity characterized in that brain wave signals containing θ wave signals, α wave signals and P wave signals during sampling time in the subjects are detected, the α wave signals and the β wave signals are separated from the brain wave signals, the integration values of the α wave signals and the integration values of the β wave signals are integrated, the integration ratio of the integrated β wave signals to the integrated α wave signals is calculated, the distribution of the occurrence frequency of the integration ratio is calculated, and the brain wave activity is judged based on this distribution of the occurrence frequency of the integration ratio.

23. A brain wave activity quantification measurement equipment characterized in that comprising a detection device detecting brain wave signals containing θ wave signals, α wave signals and β wave signals during sampling time in the subjects, the separators separating the α wave signals and the β wave signals from the brain wave signals, the integrators integrating the α wave signals and the β wave signals, a calculator calculating the integration ratio of the integrated β wave signals to the integrated α wave signals, a contour device counting the distribution of the occurrence frequency of the integration rate.

24. A brain wave activity quantification measurement equipment characterized in that comprising an amplifier extracting brain wave signals containing the dominant brain wave signals and the separators separating the α wave signals and the β wave signals from the brain wave signals, an A/D converter digitizing the brain wave signals, the α wave signals and the β wave signals that are extracted, the integrator integrating in the sampling integration time the brain wave signals, the α wave signals and the β wave signals which are converted by the A/D converter, the calculator calculating the ratio of the integration values of the β wave signals to the integration values of the α wave signals, calculating the occurrence ratio of the integration value of the α wave signals to the integration value of the wave signals (α%), calculating the occurrence ratio of the integration value of the β wave signals to the integration value of the wave signals (P%) and calculating the ratio with β% to α% to obtain the information for judgment of the brain wave activity, the memory memorizing the calculation program and the results of the calculating, and the display displaying the results of calculation.

25. A brain wave activity quantification measurement equipment as claimed in claim 5, characterized in that comprising a programming device calculating the integration values ΣS2, ΣSα2, ΣSβ2, Σα2/ΣS2=α%, Σβ2/ΣS2=β% that are integrated in set sampling integration time t, calculating the average values of Σα%/N=α3, Σβ%/N=β3, β3/α3=AW (awaking index) during the sampling period T included the sampling cycles N, and these calculations operated after the digitized procedures of the brain wave signals S, the α wave signals and the β wave signals to obtain the information for judgment of the brain wave activity.

26. A judgment method of the brain wave activity as claimed in claim 1, characterized in that the information for judgment of the brain wave activity is applied to the diagnosis help information of dementia and other mental disorders.
